(19) United States
(12) Patent Application Publication
(10) Pub. No.: US 2020/0222221 A1
(43) Pub. Date: Jul. 16, 2020

(54) GASTROINTESTINAL DEVICE DELIVERY SYSTEMS AND METHODS OF USE THEREOF

(71) Applicant: GI Dynamics, Inc., Boston, MA (US)

(72) Inventors: Scott SCHORER, Duxbury, MA (US); Chris THOMPSON, Needham, MA (US)

(21) Appl. No.: 16/632,687

(22) PCT Filed: Jul. 23, 2018
(86) PCT No.: PCT/US2018/043341

§ 371 Date: Jan. 21, 2020

Related U.S. Application Data
(60) Provisional application No. 62/535,568, filed on Jul. 21, 2017.

(51) Int. Cl. A61F 5/00

(52) U.S. Cl. CPC A61F 5/0089; A61F 5/0076

(57) ABSTRACT

The invention features delivery systems for delivering a gastrointestinal device into the gastrointestinal tract of a patient and anchoring the gastrointestinal device with an anchor (e.g., a disintegrable anchor). Also provided are methods of delivering such gastrointestinal devices using the delivery systems described herein. The methods and delivery systems of the invention can be used for treatment of metabolic diseases, such as type 2 diabetes, non-alcoholic steatohepatitis (NASH), non-alcoholic fatty liver disease (NAFLD), obesity, and related comorbidities thereof.

### BACKGROUND OF THE INVENTION

According to the Center for Disease Control, 9.3% of the population of the United States has been diagnosed with type 2 diabetes or is predicted to develop type 2 diabetes, over half of whom are clinically obese. Type 2 diabetes and obesity can be broadly characterized as metabolic disorders, that often lead to life-threatening comorbidities including non-alcoholic steatohepatitis (NASH), non-alcoholic fatty liver disease (NAFLD), hypertension, coronary artery disease, hypercholesteremia, sleep apnea, and pulmonary hypertension.

Patients suffering from metabolic diseases typically have an aberrant physiological response to ingested food after a meal. In particular, inadequate secretion of insulin has been associated with development of metabolic disorders such as type 2 diabetes. This blunted insulin response is caused by a loss or reduction of the “incretin effect,” the gut-dependent secretion of incretins (e.g., hormones such as glucagon-like peptide-1 (GLP-1) and glucose-dependent insulinotropic polypeptide (GIP)). Thus, the modulation of signaling pathways in the gastrointestinal tract is emerging as a promising approach for treating metabolic disorders, such as type 2 diabetes, obesity, and related comorbidities.

Many conventional treatments involve surgical modification of gastrointestinal anatomy. Such procedures include, for example, gastric remodeling and gastric bypass. Unfortunately, the morbidity rate for surgical procedures is alarmingly high, with 11% of cases requiring surgical intervention for correction. Early small bowel obstruction has been estimated to occur at a rate of between 2-6% in these surgeries, and mortality rates are reported to be approximately 0.5-1.5%, and are most likely much higher. While invasive surgery seems to be effective when successfully performed, the associated complication rates are unacceptably high. Laparoscopic techniques adapted to these procedures provide fewer surgical complications but continue to expose these patients to high operative risk in addition to requiring an enormous level of skill by the surgeon.

Temporary gastrointestinal devices, such as gastrointestinal sleeves, can treat metabolic disorders through incretin modulation by creating a barrier between ingested material (e.g., chyme) and a region of the gastrointestinal luminal wall (e.g., a region distal to the pylorus, e.g., duodenum and proximal jejunum). After a therapeutic period, such gastrointestinal devices need to be removed. Failure to timely remove a device can lead to adverse events.

Thus, there is a need in the field for a delivery system for securing a gastrointestinal device within a patient's gastrointestinal tract that does not require a separate procedure for removal of the device.

### SUMMARY OF THE INVENTION

A delivery system for delivering a flanged gastrointestinal device is disclosed. The delivery system enables delivery of a gastrointestinal device (e.g., a gastrointestinal sleeve) to the gastrointestinal tract (e.g., pylorus or duodenum), such that the device is automatically removed after a period of time, e.g., by disintegration of an anchoring system, or can be removed using an endoscope after a period of time. The invention also includes methods of using the delivery system, e.g., as a treatment for metabolic diseases.

The present invention provides a delivery system for placing a gastrointestinal device within the gastrointestinal tract of a patient, the delivery system including a delivery catheter including a lumen defining a longitudinal axis, wherein the delivery catheter has a distal end and a proximal end, a container assembly connected to the distal end of the delivery catheter, the container assembly including a plurality of actuator legs circumferentially arranged about the longitudinal axis, wherein each of the actuator legs includes an anchor, whereupon application of a force from the delivery catheter, the plurality of actuator legs opens outward about the longitudinal axis. In some embodiments, the delivery system further includes a gastrointestinal device. The disclosed gastrointestinal device includes a gastrointestinal sleeve having a proximal end and a distal end, the gastrointestinal sleeve configured to carry fluid from the proximal end to the distal end; and a flange connected to the gastrointestinal sleeve at or near the proximal end, the flange configured to secure the proximal end of the sleeve within the gastrointestinal tract. In the present invention, the gastrointestinal device is configured to be housed within the container assembly and pre-assembled to the delivery legs for simple delivery to the gastrointestinal tract.

Any suitable number of actuator legs may be used. In some embodiments, the number of actuator legs is equal to the number of anchors, each actuator leg containing or attached to an anchor. In some embodiments, the number of actuator legs and/or anchors is at least two (e.g., at least three, at least four, at least five, at least six, at least seven, at least eight, at least nine, at least ten, at least eleven, or at least twelve).

In some embodiments, the gastrointestinal sleeve is everted such that the flange is at or near the distal end of the container assembly, and the remainder of the sleeve is loaded within the circumferential arrangement of actuator legs (e.g., within a capsule that can be progressed through the gastrointestinal tract by normal peristalsis. In some embodiments, the anchors (e.g., disintegrable anchors) and/or actuator legs are pre-assembled through the flange, such that the flange is positioned at and secured to the gastrointestinal lumen (e.g., at or proximal to the pylorus) in one step.

In some embodiments, the actuator legs are housed within the container assembly. In further embodiments, the container assembly includes an ejection system whereupon distal displacement along the longitudinal axis, the actuator legs open outward about the longitudinal axis. In one embodiment, the ejection system is a plunger. In one embodiment, the plunger is configured to apply a distal longitudinal force on the actuator legs. In some embodiments, the plunger ejects the flange of the gastrointestinal device after outwardly opening the actuator legs. In some embodiments, the force generated by outwardly opening the actuator legs ejects the flange of the gastrointestinal device.

In some embodiments, the actuator legs are attached to distal portion of the container assembly by an articulating interface. In certain embodiments, the articulating interface is selected from the group hinges, bearings, ball joint, spring, foam, elastomer, or sponge. In some embodiments, the plunger ejects the flange of the gastrointestinal device after outwardly opening the actuator legs.

In one embodiment, the actuator legs are formed from a shape memory material, in particular nitinol. In further embodiments, the actuator legs have a physical stop such that the latitudinal dimension of the opening of the actuator legs does not exceed any latitudinal dimension of the flange.

In further embodiments, the actuator legs have a lumen. In one embodiment, the anchors (e.g., disintegrable anchors) are releasably housed within the lumen of the actuator legs. In further embodiments, the actuator legs have an anchor ejection system configured to release the anchors (e.g., disintegrable anchors), in particular a plunger. In some embodiments, the anchors (e.g., disintegrable anchors) are connected to the actuator legs chemically. In some embodiments, the anchors (e.g., disintegrable anchors) are connected the actuator legs mechanically. In some embodiments, the force applied to one or more of the anchors from the delivery system is at is between about 0.1 and 10 Newtons (N), e.g., from 0.1 N to 0.5 N, from 0.5 N to 1.0 N, from 1.0 N to 2.0 N, from 2.0 N to 3.0 N, from 3.0 N to 4.0 N, from 4.0 N to 5.0 N, from, from 5.0 N to 6.0 N, from 6.0 N to 7.0 N, from 7.0 N to 8.0 N, from 8.0 N to 9.0 N, or from 9.0 N to 10 N, e.g., about 0.1 N, about 0.5 N, about 1.0 N, about 2.0 N, about 3.0 N, about 4.0 N, about 5.0 N, about 6.0 N, about 7.0 N, about 8.0 N, about 9.0 N, or about 10 N.

In some embodiments, the flange is configured to be positioned proximal to the patient's pyloric orifice (e.g., at the antrum of the stomach, e.g., at a distal portion of the antrum).

In certain embodiments, the anchors are disintegrable. In some embodiments, the disintegrable anchors are formed wholly or partially from a degradable material. In some embodiments, the disintegrable anchors are poly(lactic acid) (PLA), poly(glycolic acid) (PGA), poly(L-lactic acid) (PLLA), poly-LD-lactide (PLDLA), poly-DL-lactide (PDLLA), diolamine, trimethylene carbonate, caprolactone, dioxanone, polydioxanone (PDO), and copolymers thereof, in particular PLLA.

In other embodiments, the anchors are not disintegrable material. In some embodiments, the non-disintegrable anchors are polyethylene terephthalate (PET), polyether ether ketone (PEEK), polyoxymethylene, polytetrafluoroethylene (PTFE), or a ceramic.

In some embodiments, the anchors (e.g., disintegrable anchors) are pre-strung with a disintegrable tether. In one embodiment, the disintegrable tether is the same material as the disintegrable anchors, in particular PLLA. In one embodiment, the tether is a different material than the anchors (e.g., disintegrable anchors). In one embodiment, the disintegrable tether is a non-degradable material selected from stainless steel, CoCr alloys, nitinol, and tantalum, in particular nitinol.

In some embodiments, the flange is circular. In some embodiments, the flange is non-circular. For example, the flange can extend around the full circumference of the sleeve and can have a substantially constant radius. In some embodiments, the maximum outer diameter of the flange is at least 10% greater than a diameter of the sleeve (e.g., about 10% greater than, about 11% greater than, about 12% greater than, about 13% greater than, about 14% greater than, about 15% greater than, about 20% greater than, about 25% greater than, about 30% greater than, any percentage between these numbers, or more, e.g., from 10% to 1,000%, from 20% to 500%, from 30% to 200%, from 40% to 100%, from 50% to 80%, or more).

In some embodiments, the diameter of the sleeve is substantially constant along its length and may have a length of at least 30 cm (e.g., about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 100 cm, about 110 cm, about 120 cm, about 130 cm, about 140 cm, or about 150 cm). In some embodiments, the diameter of the sleeve is substantially constant along its length and may have a length of no more than 150 cm (e.g., no more than 140 cm, no more than 130 cm, no more than 120 cm, no more than 110 cm, no more than 100 cm, no more than 90 cm, no more than 80 cm, no more than 70 cm, no more than 60 cm, no more than 50 cm, no more than 40 cm, or no more than 30 cm, e.g., about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 100 cm, about 110 cm, about 120 cm, about 130 cm, about 140 cm, or about 150 cm).

In some embodiments of any of the preceding aspects of the invention, the sleeve is a polymeric liner. In some embodiments of any of the preceding aspects of the invention, the polymeric liner is polytetrafluoroethylene (PTFE), expanded polytetrafluoroethylene (ePTFE), fluorinated ethylene propylene (FEP), perfluoroalkoxy (PFA), ethylene tetrafluoroethylene (EFTE), and polyvinylidene fluoride (PVDF), in particular PTFE.

Another aspect of the invention provides a method of delivering a gastrointestinal device to the gastrointestinal tract of a patient, the method having the sequential steps of providing a delivery system including a container assembly connected to a delivery catheter, the delivery catheter defining a longitudinal axis, the container assembly housing a plurality of actuator legs about the longitudinal axis, wherein the actuator legs include or are attached to an anchoring system for anchoring a distal end of the gastrointestinal device (e.g., at its flange). In some embodiments, the method includes directing the container assembly into the patient's gastrointestinal tract to a point proximal to the pylorus (e.g., within the antrum of the stomach) using the delivery catheter, opening the actuator legs outward about the longitudinal axis to position the anchoring system proximal to the pyloric sphincter, ejecting the flange from the container assembly, and ejecting the anchoring system from the actuator legs to secure the gastrointestinal device to the luminal wall.

In another aspect, the invention provides a method of delivering a gastrointestinal device to the gastrointestinal tract of a patient, the method having the sequential steps of providing a delivery system including a container assembly connected to a delivery catheter, the delivery catheter defining a longitudinal axis, the container assembly housing an anchoring system for anchoring a gastrointestinal device including a flange at its distal end, directing the container assembly into the patient's gastrointestinal tract to a point proximal to the pylorus using the delivery catheter, and ejecting the anchoring system from the container assembly to secure the gastrointestinal device to the luminal wall.

In a further aspect, the invention provides a method of treating a metabolic disorder, the method including delivering any of the gastrointestinal devices described herein, and/or using any of the methods of delivery described herein.

### BRIEF DESCRIPTION OF THE DRAWINGS

The following drawings are illustrative of particular embodiments of the present invention and are not limiting to various embodiments encompassed by the present invention.

FIG. 1 is a drawing of the delivery of a flanged gastrointestinal device to the pylorus using a delivery system of the invention.

FIG. 2 is a drawing of an embodiment of a disintegrable anchor with two tissue retention elements projecting outward from the main body of the anchor. The embodiment further shows an example of an eyelet as the protruding portion.

FIG. 3 is a drawing of an embodiment of a tether passing through the eyelet of an anchor which has been inserted through the horizontal plane of the flange.

FIG. 4 is a drawing of an embodiment of a flange of a gastrointestinal device for securing to the pyloric sphincter using the anchoring system of the invention showing reinforced apertures.

FIG. 5 is a drawing of an embodiment of a complete anchoring system for a flanged gastrointestinal device, showing a plurality of anchors passed through the apertures of a flange and all anchors connected through their eyelets with a tether.

FIGS. 6A-6E are drawings of the sequential steps for delivering a gastrointestinal device using an embodiment of a delivery system of the invention. FIG. 6A shows the gastrointestinal device contained within the container assembly with the actuator legs closed. FIG. 6B shows the opening of the actuator legs to outwardly expand the flange of the gastrointestinal device. FIG. 6C shows the plunger direction though the container assembly. FIG. 6D shows the plunger forcing the everted gastrointestinal device out of the container assembly towards the pylorus. FIG. 6E shows the plunger forcing the everted gastrointestinal device through the pylorus and into the duodenum.

### DETAILED DESCRIPTION OF THE INVENTION

The present invention provides delivery systems for delivering a gastrointestinal device into the gastrointestinal tract of a patient and anchoring the gastrointestinal device with a disintegrable anchor, obviating the need for a follow on procedure for removal of the device. Also provided are methods of delivering such gastrointestinal devices using the delivery systems described herein. The methods and delivery systems of the invention can be used for treatment of metabolic diseases, such as type 2 diabetes, non-alcoholic steatohepatitis (NASH), non-alcoholic fatty liver disease (NAFLD), obesity, and related comorbidities thereof.

### Definitions

As used herein, the terms “gastrointestinal device” includes an anchor and/or anchoring system for securely positioning the gastrointestinal device to the stomach and a sleeve to limit absorption of nutrients in the duodenum.

As used herein, a “sleeve,” refers to a hollow, cylindrical gastrointestinal liner that is open at both ends and adapted to extend longitudinally within the duodenum. Fluid (e.g., partially digested food, chyme, or other endogenous or exogenous fluid) passing through the gastrointestinal tract passes through the interior of the sleeve.

As used herein, a “flanged” element or a “flange” refers to a projection that, when implanted in a subject, wholly or partially extends radially (i.e., in a direction having a radial component from a longitudinal axis of a pyloric orifice (e.g., between 90° and 180° outward from the longitudinal axis of the pyloric orifice)) and configured to attach a gastrointestinal device to a proximally oriented luminal surface (e.g., a proximal surface of the pyloric sphincter and/or an antral surface of the stomach).

As used herein, an “anchor” refers to an implantable element of a gastrointestinal device that resists both latitudinal and longitudinal motion (e.g., migration resulting from peristalsis or fluid shear forces) of the gastrointestinal device by having a physical connection to a gastrointestinal luminal wall.

An anchor may resist motion of the gastrointestinal device by exerting a proximal force on a distally-oriented gastrointestinal luminal wall or by exerting a distal force on a proximally oriented gastrointestinal luminal wall. Anchors may or may not penetrate a gastrointestinal luminal wall.

To “secure” an anchor or a gastrointestinal device refers to an act that results in the immobilization of the anchor or device at a position. For example, securing an anchor includes deploying an anchor such that the anchor penetrates the tissue of the gastrointestinal tract, thereby immobilizing a gastrointestinal device.

Unless otherwise specified, a longitudinal axis refers to the longitudinal axis of the gastrointestinal tract (i.e., the line running through the gastrointestinal lumen equidistant from the luminal walls). It will be understood that, due to the tortuosity of the gastrointestinal tract, the directionality of its longitudinal axis and associated radial coordinates will vary along its length. For cases in that the “longitudinal axis of the device” is referred to, it is explicitly referred to as such.

The orientation of any surface (e.g., a luminal surface, luminal wall, or device surface) is characterized herein according to the direction of its normal line (i.e., a vector originating at and projecting orthogonally outward from its surface). As used herein, the orientation of a gastrointestinal luminal surface is an average of any micro features and is therefore independent of, e.g., microvilli.

The angle between a normal line and the longitudinal axis of a “proximally oriented” or a “distally-oriented” surface is ≥0° and <90°. For example, a “proximally oriented surface” of a lumen herein refers to a luminal surface that faces in a proximal direction (e.g., the stomach side of the pyloric sphincter), whereas a “distally-oriented surface” of a lumen herein refers to a luminal surface that faces in a distal direction (e.g., the intestinal side of the pyloric sphincter).

The term “pyloric orifice” refers to open area on the plane in that the pyloric opening lies.

The term “pyloric sphincter” refers to the tissue (e.g., epithelial and muscular tissue) that surrounds the pyloric orifice.

As used herein, “outward” refers to any direction that is substantially orthogonal to a reference longitudinal axis (e.g., the longitudinal axis of the anchor). For example, actuator legs open away from the longitudinal axis that they are aligned on when housed in a container assembly.

As used herein, the term “disintegrable” refers to any material that is capable being broken into one more segments. The material can be broken by physiological conditions within the body, e.g., by digestive mechanical action, e.g., peristalsis, or by chemical interactions, e.g., pH, hydrolysis, or other biochemical reactions within the gastrointestinal tract. The disintegration of the material may be controlled by the molecular weight of the material, the physical dimensions, e.g., length, width, or mass, of the object that is to be dissolved over a fixed or variable length of time installed in the body.

As used herein, the term “degradable” refers to any material that is capable being dissolved under normal physiological conditions and passed through the body. The degradation of the material may be controlled by the molecular weight of the material, the physical dimensions, e.g., length, width, or mass, of the object that is to be dissolved over a fixed or variable length of time installed in the body, or the local environment within the body.

As used herein, the term “resorbable” refers to any material that is capable being absorbed into the body after dissolution under normal physiological conditions.

As used herein, the term “about” refers to +/−10% of a recited value.

As used herein, the term “treatment” refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, the gastrointestinal device is used to control metabolic disorders (e.g., type 2 diabetes, NASH, NAFLD, obesity, and related comorbidities). In some embodiments, removal of gastrointestinal device is provided to delay development of a disease or to slow the progression of a disease.

As used herein, the terms “subject” or “patient” refer to any mammal (e.g., a human) having a gastrointestinal tract capable of containing of gastrointestinal device of the invention. A patient who is being treated for a metabolic disorder, e.g., high blood sugar, diabetes (e.g., type 2 diabetes), obesity, NASH, NAFLD, or a related comorbidity thereof, may be one who has been diagnosed by a medical or veterinary clinician as the case may be as having such a condition. Diagnosis may be performed by any suitable means. Patients of the invention may have been subjected to standard tests or may have been identified, without examination, as one at high risk of having or developing a metabolic disorder, e.g., type 2 diabetes, obesity, NASH, NAFLD, or a related comorbidity due to the presence of one or more risk factors, such as age, genetics, or family history.

As used herein, the term “comorbidity” or “related comorbidity” refers to one or more conditions, syndromes, diseases, or disorders that co-occur with metabolic disorders and can be either directly or indirectly linked to metabolic disorders. For example, metabolic disorder-related conditions may include type 2 diabetes, obesity, NAFLD, NASH, dyslipidemia, elevated serum/plasma LDL, elevated VLDL, elevated triglycerides, elevated cholesterol, plaque formation leading to narrowing or blockage of blood vessels, glucose intolerance, myocardial infarction, increased risk of hypertension/stroke, or coronary heart disease. As used herein, “diabetes mellitus type 2” or “type 2 diabetes” (also known as diabetes mellitus type 2, non-insulin-dependent diabetes (NIDDM), obesity-related diabetes, or adult-onset diabetes) refers to a metabolic disorder that is primarily characterized by insulin resistance, relative insulin deficiency, and hyperglycemia.

As used herein, the term “gastrointestinal,” “GI,” “gastrointestinal tract,” or “GI tract” refer to the entire alimentary canal, from the oral cavity to the rectum (e.g., the tube that extends from the mouth to the anus, including, e.g., the esophagus, stomach, small intestine, large intestine, and rectum) in that the movement of muscles and release of hormones and enzymes digest food.

As used herein, the term “incretin” refers to a compound that directly or indirectly stimulates insulin release, inhibits glucagon release, and reduces gastric emptying. For example, incretins stimulate an increase in the amount of insulin released from the pancreas when plasma glucose levels are elevated relative to normal after food consumption, thereby leading to a decrease in blood glucose levels. Specific examples of incretins include gastric inhibitory peptide (i.e., glucose-dependent insulinotropic polypeptide, or GIP) and glucagon-like peptide-1 (GLP-1), along with their analogs and derivatives.

As used herein, “sleeve-coupling interface” refers to the point of the sleeve that is connected to anchor by a coupling liner. In some cases, the sleeve-coupling interface circumscribes the sleeve at a point along its length, e.g., at or distal to the distal end of the anchor.

As used herein, the term “fluid” refers to digested or partially digested material, for example chyme and digestive secretions.

In the event of any term having an inconsistent definition between this application and a referenced document, the term is to be interpreted as defined herein.

### Delivery Systems

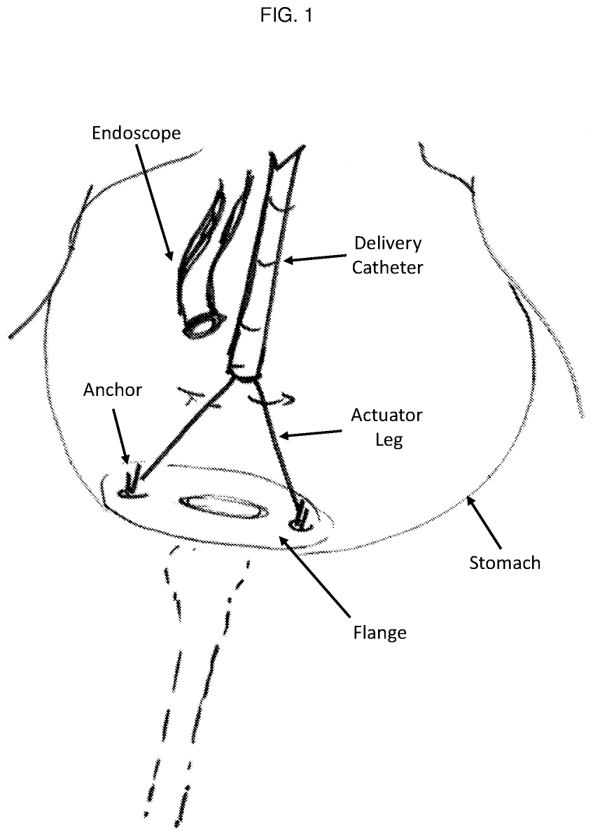

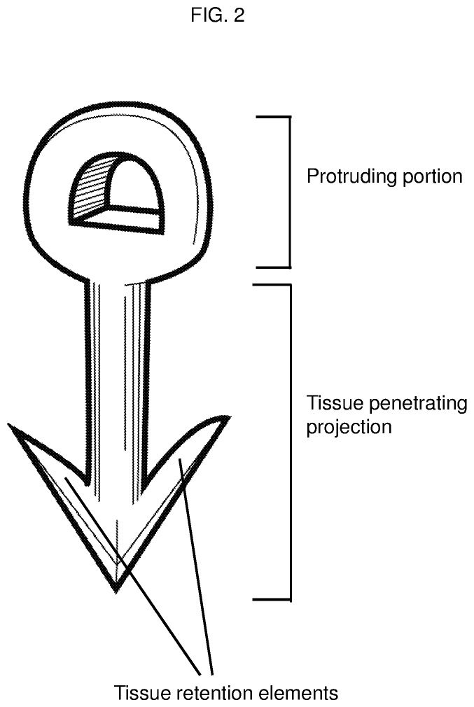

The present invention features delivery systems for placing and securing a gastrointestinal device in a patient's gastrointestinal tract. In particular, the delivery systems described herein are configured for anchoring a gastrointestinal sleeve at or distal to the patient's pylorus using a partially disintegrable anchoring system, obviating the need for a follow-up operation to remove the device. Delivery systems include a delivery catheter having, attached to its distal end, a container assembly for housing a gastrointestinal device (e.g., all or a portion of the gastrointestinal device, including a gastrointestinal sleeve). The gastrointestinal device itself may be included in the delivery system or separate from the delivery system. A general schematic of one embodiment of the delivery system is shown in FIG. 1. For example, one embodiment of the delivery system may be compatible with various types of gastrointestinal devices (e.g., a gastrointestinal device having a wave anchor or transpyloric element, such as a flange), as a modular system. In general, the container assembly includes a plurality of actuator legs arranged around the longitudinal axis defined by the delivery catheter. Each actuator leg includes an anchor (e.g., as shown in FIG. 2) to be inserted into a tissue plane proximal to the pylorus. The actuator legs are configured such that, upon the container assembly reaching a target position (e.g., proximal to the pylorus), they may be outwardly opened by an ejection mechanism. Upon opening, the actuator legs position the anchors around the pyloric orifice in the antrum of the stomach in preparation for anchoring the gastrointestinal device in place. In some instances, a flange of the gastrointestinal device and the anchors are pre-assembled, such that the gastrointestinal device is delivered and anchored in the same step, and the remainder of the gastrointestinal device (e.g., the sleeve) is extended into the gastrointestinal tract (e.g., duodenum and beyond, e.g., the upper jejunum).

### Delivery System Components

The delivery system assembly includes the container assembly connected, at a proximal region (e.g., at its proximal end or a proximal region within the interior of the container assembly) to a delivery catheter. Delivery catheters include one or more longitudinal lumens and are described in detail in U.S. Pat. No. 7,837,643, incorporated herein by reference. Container assemblies of the present invention are configured to house a gastrointestinal device for delivery to a region of the gastrointestinal tract, e.g., by ejecting the gastrointestinal device such that the gastrointestinal device is untethered from the container assembly. Such systems and methods of use thereof are described in International Patent Application No. PCT/US2017/015658, incorporated herein by reference. Additionally or alternatively, the gastrointestinal device may be ejected simultaneously with the opening of the actuator legs. The container assembly is sized and shaped to house the gastrointestinal device (e.g., a compressed gastrointestinal device) while being suitable for delivery through a patient's mouth and gastrointestinal system (e.g., to the pylorus). Thus, in some cases, the container assembly is less than 30 mm in width at one or more directions perpendicular to the longitudinal axis (e.g., diameter; e.g., less than 30 mm, less than 29 mm, less than 28 mm, less than 27 mm, less than 26 mm, less than 25 mm, less than 24 mm, less than 23 mm, less than 22 mm, less than 21 mm, less than 20 mm, less than 19 mm, less than 18 mm, less than 17 mm, less than 16 mm, less than 15 mm, less than 14 mm, less than 13 mm, less than 12 mm, less than 11 mm, less than 10 mm, less than 9 mm, less than 8 mm, less than 7 mm, less than 6 mm, or less than 5 mm; e.g., from 5 mm to 30 mm, from 8 mm to 25 mm, from 10 mm to 20 mm, or from 12 mm to 15 mm in width at one or more directions perpendicular to the longitudinal axis (e.g., diameter)).

The ejection mechanism for delivering the gastrointestinal device from the container assembly may be a plunger operatively controlled via a wire terminating at the proximal end of the container assembly at a pusher plate. The pusher plate has a distally-oriented surface configured to exert a distal force on the actuator legs contained within or distal to the container assembly and the gastrointestinal device (e.g., a proximal end of the gastrointestinal device, e.g., a proximal end of an anchor) to deploy the gastrointestinal device. Alternatively, the ejection mechanism may include a distal element other than a plate, such as a ring, or a non-circular element having a suitable geometry and mechanical properties to displace the gastrointestinal device.

The distal region of the ejection mechanism (e.g., the pusher plate) may be articulated by a clinician through a rigid pusher wire attached thereto. The pusher wire may run longitudinally through a lumen of the delivery catheter to a proximal region (e.g., a proximal end) of the delivery catheter. Optionally, the ejection mechanism may include a mechanism to prevent a clinician from directing the pusher plate distally to such an extent that the pusher plate emerges from the distal end of the container assembly. Such mechanisms involve a moving stop and/or a static stop displaced proximally to the pusher plate, and are described in detail in U.S. Pat. No. 7,837,643, incorporated herein by reference.

### Actuator Legs

Gastrointestinal devices of the invention are configured to be released from the delivery system and/or anchored proximal to the pylorus using a plurality of actuator legs to outwardly open the flange of the gastrointestinal device once guided to a location proximal to the pylorus. The number of actuator legs is dependent on the number of anchors to be used to secure the gastrointestinal device within the gastrointestinal tract. In some cases, the number of actuator legs ranges from two to twelve (e.g., from four to eight, e.g., two, three, four, five, six, seven, eight, nine, ten, eleven, or twelve).

The actuator legs may be configured to contain the anchoring system of the gastrointestinal device and are configured to transmit a force necessary to secure the gastrointestinal device to the tissue proximal to the pylorus.

In some cases, the actuator legs are arranged circumferentially within the container assembly about the longitudinal axis formed by the delivery catheter. The actuator legs may be pre-shaped before assembling the container to have a precisely controlled outer dimension (e.g., radial width) when opened from the container assembly. The longitudinal length of the actuator legs, in addition to the shape, will exert some control over the outward expansion of the actuator legs. In some cases, the actuator legs have a longitudinal length between 10 mm and 50 mm (e.g., between 15 mm and 45 mm, between 20 mm and 40 mm, 25 mm and 35 mm, e.g., about 10 mm, about 15 mm, about 20 mm, about 25 mm, about 30 mm, about 35 mm, about 40 mm, about 45 mm, or about 50 mm).

In other embodiments, the actuator legs may be affixed to the distal edge of the container assembly using an articulating interface. The articulating interface allows the actuator legs to be opened to a fixed outward dimension and return to their original position. For example, the actuator legs may be attached to a hinge, bearings, ball joint, spring, foam, elastomer, or sponge as the interface to the distal end of the container, as all of these materials flex in response to an external force and can be configured to return to their original position upon removal of that force. The actuator legs may further include a physical stop mechanism, such as a dent, notch, or raised portion, e.g., a bump or nodule, of material along the length of the actuator leg. The physical stop contacts the distal edge of the container assembly to limit the outward opening of the actuator legs beyond a specified dimension, e.g., no larger than any one dimension, e.g., latitudinal, of the flange, so as to not tear the flange before or during the installation procedure.

When the actuator legs are configured to be housed within the container assembly, the actuator legs may be formed from a flexible material that can be deformed but return to an original shape upon reduction of an applied force. For example, the actuator legs may be formed from a shape memory metal alloy, such as spring steel, alloys of copper-aluminum-nickel, nitinol (NiTi), or shape memory polymer composites (e.g. polyurethane, polynorbornene). When the actuator legs are configured to be attached to the distal end of the container assembly using an articulating interface, the actuator legs may be formed from any suitable semi-rigid or rigid material, including the shape memory materials described herein, stainless steel or other metals, other polymers (e.g., polyethylene terephthalate (PET)), polyether ether ketone (PEEK), or ceramics. An exemplary shape memory material for the actuator legs is nitinol. Other semi-rigid or rigid materials for use in the human body are known in the art.

The actuator legs can be opened outwardly to any suitable dimension (e.g., radial width) to span the pyloric sphincter. In some embodiments, the actuator legs are opened to a radial width between 10 mm and 80 mm (e.g., between 20 mm and 70 mm, or between 30 mm and 60 mm, e.g., about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm or about 80 mm). In some embodiments, the operator is able to control the degree of outward opening of the actuator legs to suit an individual patient's anatomy, for example, by turning a knob, crank, or wheel at the proximal end of the delivery catheter. For example, actuator legs housed within the container assembly can be configured to outwardly open to an extent corresponding to their distal displacement from the distal opening of the container assembly.

In various embodiments, the actuator legs are configured to release the anchoring system for the gastrointestinal device being implanted within a patient. The anchoring system may be attached to the exterior surface or the tip of the actuator legs using a releasable chemical adhesive (such as fibrin, mixtures of gelatin and thrombin, or other viscous polymers (e.g., polyethylene glycol (PEG)). Other chemical adhesives suitable for use within the human body are known in the art. Alternatively, the anchoring system may be attached to the exterior of the actuator legs using mechanical means, such as connecting the anchoring system to the actuator leg with a releasable portion of a suitable material. For example, the anchoring system and actuator legs can be connected together with a length of a thin polymer, e.g., a band, strip, strap, or ribbon, such as polytetrafluoroethylene (PTFE) or other similar material. In any of the above embodiments, the attachment between the actuator legs and anchoring system should to be sufficiently strong to resist breakage during delivery of the gastrointestinal device to the target area (e.g., proximal to the pylorus) but releasable upon securing of the anchors and the device (e.g., the force required to release the anchors from the actuator legs does not exceed the strength of the attachment of the anchoring system to the target tissue upon the upward distal force of removing the delivery system).

In some cases, the actuator legs may be formed from a hollow piece of the actuator leg material described herein such that the anchoring system fits within the lumen of the actuator leg and may be released upon application of a sufficient distal force. The inner dimension(s) (e.g., diameter) of the lumen of the leg may be chosen such that the anchors (e.g., disintegrable anchors) of the anchoring system may be released with a low coefficient of friction. When the actuator legs are in this configuration, the anchors (e.g., disintegrable anchors) of the anchoring system may be ejected with an ejection mechanism including a pusher plate and a pusher wire, similar to the ejection system of the container assembly described herein. The pusher plate may have a distally-oriented surface configured to exert a distal force on the anchoring system contained within each actuator leg to force it out of the actuator leg and into the direction of insertion into the target area. The distal pusher plate may be of any suitable geometry that can transmit a sufficient distal force to the anchoring system contained within the actuator leg, including a ring or, alternatively, a non-circular shaped element. The pusher wire may run longitudinally through the main lumen of the delivery catheter or may be run through a separate lumen within the delivery catheter.

The force applied to the anchors to secure the gastrointestinal implant to a tissue may depend on various factors, such as the size and shape of the anchor (e.g., the tissue penetrating projection of the anchor). In some cases, the force applied to the tissue penetrating projection of the anchors is between about 0.1 Newtons and 10 Newtons (e.g. between about 0.5 and about 8 N, between about 1 N and about 6 N, or between about 2 N and about 4 N, e.g., about 0.1 N, about 0.2 N, about 0.3 N, about 0.4 N, about 0.5 N, about 0.6 N, about 0.7 N, about 0.8 N, about 0.9 N, about 1 N, about 2 N, about 3 N, about 4 N, about 5 N, about 6 N, about 7 N, about 8 N, about 9 N, or about 10 N; e.g. 0.1 to 3.4 N, 0.1 N to 1.3 N, or 3.3 N to 5.9 N).

### Anchoring Systems

In general, anchors of the present invention are small fasteners configured to have all or a portion of the fastener enter a biological tissue and resist being removed under applied mechanical stresses (e.g., proximal forces). Anchors of the present invention feature a projection that is able to penetrate the tissue a device is being attached to, e.g., the pyloric sphincter, and portion that protrudes above the tissue plane once the anchor penetrates. In some cases, the tissue penetrating projection of the anchor may have a longitudinal length of about 1 mm to about 20 mm (e.g., from about 1 mm to about 2 mm, from about 2 mm to about 4 mm, from about 4 mm to about 6 mm, from about 6 mm to about 8 mm, from about 8 mm to about 10 mm, from about 10 mm to about 12 mm, from about 12 cm to about 14 mm, from about 14 mm to about 16 mm, from about 16 mm to about 18 mm, from about 18 mm to about 20 mm).

In order to resist removal from the tissue after insertion, the tissue penetration projection of the anchors may contain one or more tissue retention elements incorporated into the structure that are advantageous for resisting longitudinal or latitudinal forces. For example, the tip, e.g., the distal end, of the tissue penetrating projection may have a hook, a prong, ridge, e.g., helical ridge, or other mechanical fastener appropriate for retaining tissue. The tissue retention elements may be bi-directional, i.e., some retention elements are pointed in the direction of forward peristalsis to secure the anchor against forward motion through the gastrointestinal tract, and some elements are pointed opposite the direction of forward peristalsis, to secure the anchor against reverse motion in the gastrointestinal tract. In some cases, the anchors may have a single tissue retention element at the distal end of the anchor, with the distal end referring to the portion of the tissue penetrating projection that first makes contact with tissue. Additionally or alternatively, an anchor may have multiple tissue retention elements at the distal end of each anchor; an embodiment of an anchor with multiple, e.g. more than one, tissue retention element is shown in FIG. 2, which has two prongs. Further, an anchor can have tissue retention elements along the entire longitudinal length of the anchor, each being the same or different types of tissue retention elements.

Each of the anchors may further include a protruding potion that extends above the tissue plane once the tissue penetrating projection of the anchor is inserted into a tissue. In certain embodiments, the protruding portion of each anchor can have a length that is at least 5% less than the length of the tissue penetrating projection of the anchor, e.g. at least 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or more, less than the length of the tissue penetrating projection. For example, the length of the protruding portion may be from at least 2 mm to 40 mm (e.g., from 2 m to 40 mm, from 3 mm to 36 mm, from 4 mm to 32 mm, from 5 mm to 28 mm, from 6 mm to 24 mm, from 7 mm to 20 mm, from 8 mm to 16 mm, from 9 mm to 12 mm, or at least 10 mm). The protruding portion of each anchor is configured with an eyelet through which a tether can pass to connect two or more anchors together as an anchor system. The eyelet may be of any suitable profile, e.g., circular or non-circular, to allow a tether to pass with a low coefficient of friction (e.g., a static or kinetic coefficient of friction of less than about 0.3, e.g., about 0.29, about 0.28, about 0.27, about 0.26, about 0.25, about 0.24, about 0.23, about 0.22, about 0.21, about 0.20, about 0.19, about 0.18, about 0.17, about 0.16, about 0.15, about 0.14, about 0.13, about 0.12, about 0.11, about 0.10, or less). In some cases, the dimensions of the eyelet may have a larger width than height, e.g., rectangular or ovoid, rather than a circular opening. In this configuration, the overall profile of the protruding portion is minimized relative to the tissue plane.

In some cases, the anchors are configured to be disintegrable over time under normal physiological conditions. Disintegrable materials are typically degradable polymers including, but not limited to, poly(lactic acid) (PLA), poly(glycolic acid) (PGA), poly(L-lactic acid) (PLLA), poly-DL-lactide (PDLLA), poly-LD-lactide (PLDLA), diolamine, trimethylene carbonate, caprolactone, dioxanone, polydioxanone (PDO), and copolymers thereof, (e.g., poly(lactic-co-glycolic acid) (PLGA) or poly(L-Lactide-DL-lactide) (PLDL)). The typical averaged molecular weight (Mw) range for a degradable polymer useful for the anchors of the invention is about 20,000 g/mol to about 500,000 g/mol, e.g., about 20,000 g/mol to about 50,000 g/mol, about 30,000 g/mol to about 70,000 g/mol, about 40,000 g/mol to about 80,000 g/mol, about 50,000 to 100,000 g/mol, about 50,000 g/mol to about 150,000 g/mol, about 150,000 g/mol to about 250,000 g/mol, about 200,000 g/mol to about 300,000 g/mol, about 250,000 g/mol to about 350,000 g/mol, about 300,000 g/mol to about 400,000 g/mol, about 350,000 g/mol to about 450,000 g/mol, or about 400,000, to about 500,000 g/mol. Other degradable materials useful for applications within the human body are known in the art. The disintegrable material may further be resorbable, e.g. absorbed within the gastrointestinal tract after disintegrating rather than being passed with through the normal excretory path of the gastrointestinal tract. The tissue penetrating projection and the protruding portion may be made of the same disintegrable material such that both parts have identical degradation kinetics. Alternatively, the tissue penetrating projection and the protruding portion may be made of different disintegrable materials such that both parts can have different erosion kinetics with a controllable degradation time. In some embodiments, the tissue penetrating projection of the anchor may be made from a disintegrable material and the protruding portion of the anchor may be made from a non-degradable material. In this configuration, when the tissue-embedded anchor disintegrates, the eyelet is released and passes through the gastrointestinal tract. The protruding portion may be made of any suitable non-degradable material, such as stainless steel, alloys of CoCr, nitinol (NiTi), tantalum, non-degradable polymers (e.g., polyethylene terephthalate (PET)), polyether ether ketone (PEEK), or ceramics).

In some embodiments, when configured to be degradable, the anchors or tissue penetrating projections thereof are configured to completely degrade or resorb at a time point from 1 week to 1 year (e.g., from 2 weeks to 40 weeks, from 4 weeks to 35 weeks, from 8 weeks to 30 weeks, or from 10 weeks to 20 weeks, e.g., from 2 weeks to 4 weeks, from 4 weeks to 6 weeks, from 6 weeks to 8 weeks, from 8 weeks to 10 weeks, from 10 weeks to 12 weeks, from 14 weeks, from 14 weeks to 16 weeks, from 16 weeks to 18 weeks, from 18 weeks to 20 weeks, from 20 weeks to 30 weeks, from 30 weeks to 40 weeks, or from 40 weeks to 50 weeks).

Alternatively, the anchors of the anchoring system may be configured to act as a substantially permanent installation point for a replaceable gastrointestinal device, e.g., a sleeve, and these anchors may be made of a suitable biocompatible material that can resist disintegration and/or degradation under normal physiological conditions, e.g., a non-degradable polymer, e.g., polyethylene terephthalate (PET)), polyether ether ketone (PEEK), polyoxymethylene, polytetrafluoroethylene (PTFE), or ceramics. Other suitable materials are known in the art. For example, the anchors may be deployed to the operative location within the gastrointestinal tract connected to the gastrointestinal device, and at the recommended frequency, the previously installed device can be removed from the anchors and a new device connected thereto with the anchors remaining in their installed position. In this configuration, the tissue penetrating projection and the protruding portion of the anchor may be made from the same material, e.g., the anchor material, or alternatively, the tissue penetrating projection and the protruding portion may each be a different non-degradable material. The protruding portion may be made of any suitable non-degradable material, such as stainless steel, alloys of CoCr, nitinol (NiTi), tantalum, non-degradable polymers (e.g., polyethylene terephthalate (PET)), polyether ether ketone (PEEK), or ceramics), and the tissue penetrating portion may be PET, PEEK, polyoxymethylene, PTFE, or ceramic. Other suitable materials are known in the art.

Alternatively or in addition, anchors useful for the present invention may include anchors that have pivoting or compressible retention elements, such as those disclosed in U.S. Pat. Nos. 9,572,565, 9,545,255, 8,740,940, 7,416,554, 7,361,180, and 7,736,379, each of which is incorporated herein by reference. A skilled artisan can readily appreciate that a simple modification, e.g., incorporation of an eyelet or similar structure, to the referenced anchors may be used in the present invention.

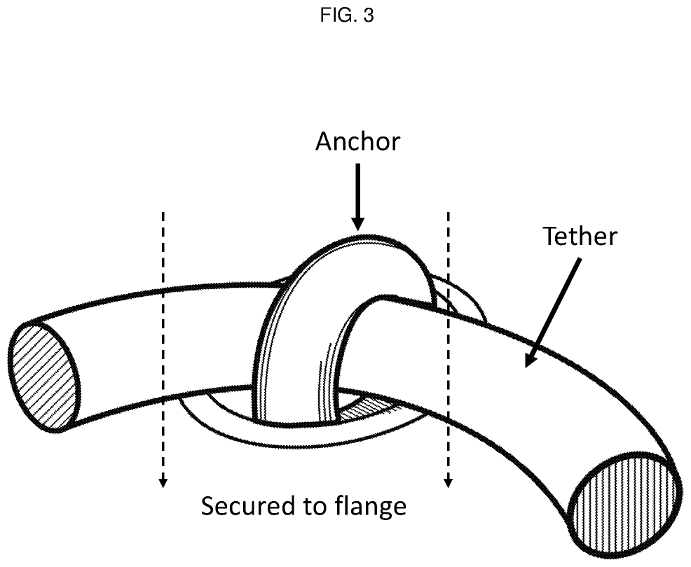

The anchoring system of the invention is configured to have the protruding portion of each anchor connected together by a disintegrable tether placed through the eyelets. An embodiment of this configuration is shown in FIG. 3. The disintegrable tether is configured to disintegrate prior to the disintegrable anchors such that when the tether disintegrates, it separates from the anchors by passing through the eyelets. The tether can then be passed through the body's normal excretory path. When the tether is no longer present, the flange of the gastrointestinal device is able to come free of the anchors by the apertures of the flange passing over the anchors. This allows the gastrointestinal device to fall away as a single piece, which minimizes the chance of injury to sensitive gastrointestinal tract tissues or the formation of a blockage from the device becoming lodged in within the gastrointestinal tract.

The tether may be made of the same disintegrable material as the disintegrable anchors as described herein. Alternatively, the tether may be made of a combination of the disintegrable material and a structural enhancing component, such as polycaprolactone (PCL). As it is designed be disintegrated prior to the anchors and is sized such that it passes through the eyelets of the anchors, the tether may be thinner than the anchors, allowing it to have faster disintegration kinetics. In some embodiments, the tether and the anchors are made of different disintegrable materials, including those described herein. In some embodiments, the tether may be made of two separate portions, with the first portion being a break point made of a disintegrable material and the second portion being a substantially non-degradable material. The tether is configured to slide through the eyelets of the anchors fall away when the disintegrable break point disintegrates, allowing the non-degradable tether to pass through the gastrointestinal tract. Other types of fastening systems, e.g., tethers, for connecting two objects together, e.g., loops, sutures, and staples, are known in the art.

The shape and physical dimensions of the tether may be chosen such that it matches the shape of the eyelet of the anchor to minimize friction as it is passed through the eyelet. In some embodiments, the tether has a cross-sectional dimension that is at least 10% less than the largest cross-sectional width of the opening of the eyelet of the disintegrable anchor (e.g., 10%, 20%, 30%, 40%, 50%, 60%, 70%, 80%, 90%, 100%, or more). For example, the tether may have a cross-sectional dimension from about 10 μm to about 1000 μm (e.g., from about 10 μm to about 1000 μm, about 20 μm to about 900 μm, about 30 μm to about 800 μm, about 40 μm to about 700 μm, about 50 μm to about 600 μm, about 60 μm to about 500 μm, about 70 μm to about 400 μm, about 80 μm to about 300 μm, about 90 μm to about 200 μm, about 100 μm).

Useful cross-sectional geometries or tethers of the anchoring system of the invention include circular and non-circular geometries. For example, the tether may be a piece of wire having a circular cross-section, e.g., a round wire. In some cases, the tether has a geometry where the width of the tether is substantially larger than its height, e.g., a rectangle or ovoid. In this configuration, as with the anchors that have a similar rectangular or ovoid opening, the longitudinal profile of the tether is minimized relative to that of a circular tether, keeping the overall height and profile on the installed anchor/tether combination at a minimum. For example, the tether whose width is substantially larger than its height may have a maximum cross-sectional width from about 10 μm to about 1000 μm (e.g., from about 10 μm to about 1000 μm, about 20 μm to about 900 μm, about 30 μm to about 800 μm, about 40 μm to about 700 μm, about 50 μm to about 600 μm, about 60 μm to about 500 μm, about 70 μm to about 400 μm, about 80 μm to about 300 μm, about 90 μm to about 200 μm, about 100 μm).

### Gastrointestinal Devices

The invention features gastrointestinal devices for limiting contact and transfer of material across luminal walls along a segment of the gastrointestinal tract (e.g., at the duodenum and/or upper jejunum). Gastrointestinal devices of the invention may include a liner (e.g., a bariatric sleeve) and a flange for attachment to a tissue proximal to the pyloric orifice (e.g., to the pyloric tissue or antrum of the stomach).

### Flanges

The invention also features a gastrointestinal sleeve that has a flanged proximal end for attaching the device to a portion of a patient's gastrointestinal tract, e.g., a proximal surface of the pyloric sphincter and/or the antral surface of the stomach. By attaching to a proximally oriented tissue surface, a flange can guide fluid (e.g., chyme) from the stomach into the sleeve while providing resistance to distal migration of the gastrointestinal device, e.g., as a result of peristalsis. The flange of the sleeve may be configured to be anchored to the pyloric sphincter by having a plurality of apertures through the flange sized such that an anchor can pass though aperture and thus the plane formed by the flange's surface. The apertures of the flange may be of any suitable shape, e.g., holes, slits, or slots, such that the anchors can pass through the aperture with a low coefficient of friction.

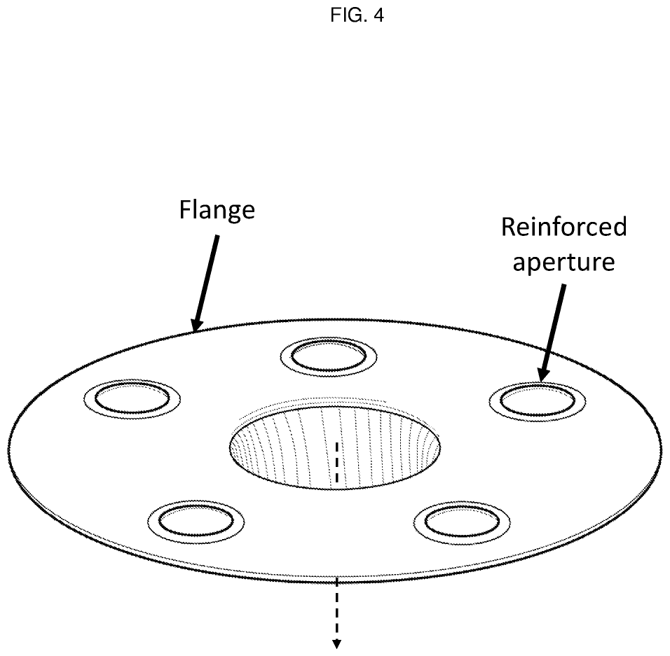

In order to increase the durability of the flange and to minimize the change of the tearing the flange after installing the gastrointestinal device or during a period of treatment, each of the apertures in the flange may be reinforced (e.g., have increased durability and/or thickness) relative to the rest of the flange material. The reinforcement for the apertures may be made of the same material as the flange, or may be made of a different material with different reinforcement properties, such as a different polymer or metal. An exemplary flanged gastrointestinal device with reinforced apertures is shown in FIG. 4

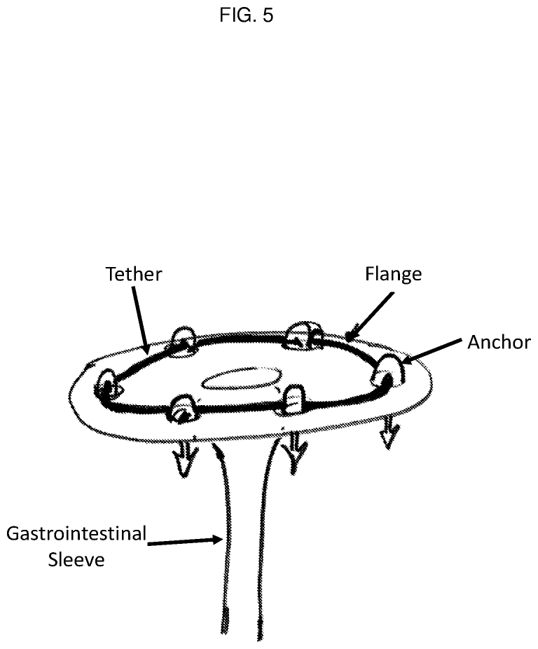

An example of a complete anchoring system for a flanged gastrointestinal device is shown in FIG. 5. In general, a flange is attached to a proximal portion of the sleeve. In some cases, the flange defines the proximal opening of the sleeve (e.g., as a circular “rim” or “skirt” connected to the proximal end of the sleeve). In some embodiments, the flange extends radially in all directions from the sleeve, helping prevent fluid from passing around the outside of the proximal end of the sleeve. The outer diameter of the flange may be constant (e.g., substantially circular). In such cases, the outer diameter of the flange can be at least 10% greater than the diameter of all or a portion of the sleeve (e.g., the proximal end of the sleeve). In some cases, the outer diameter of the flange is about 6-10 cm. In some embodiments, the outer diameter of the flange is about 6 cm, about 7 cm, about 8 cm, about 9 cm, or about 10 cm. The length of the sleeve extending from the distal end of the flange to the proximal end of the anchor ranges from about 1 cm to about 10 cm. In some embodiments, the length of the sleeve extending from the distal end of the flange to the proximal end anchor is about 1 cm, about 2 cm, about 3 cm, about 4 cm, about 5 cm, about 6 cm, about 7 cm, about 8 cm, about 9 cm, or about 10 cm. In some cases, the length of the sleeve extending from the distal end of the flange to the proximal end of the anchor is 3 cm. In other cases, the length of the sleeve extending from the distal end of the flange to the proximal end of the anchor is 6 cm. The hole in the flange can be suitable to accommodate the diameter of the sleeve (e.g., from 1 to 3 cm, e.g., about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, about 2.5 cm, about 2.6 cm, about 2.7 cm, about 2.8 cm, about 2.9 cm, or about 3.0 cm).

Alternatively, the flange may extend radially at different lengths around the circumference of the sleeve (e.g., its outer diameter may be variable, e.g., ovoidal or irregularly shaped). Such a configuration may be adapted for an asymmetric anatomy and/or physiological forces (e.g., at the pylorus or antrum of the stomach). Additionally or alternatively, an irregularly shaped outer diameter of a flange may serve to optimize the seal of the flange to the gastrointestinal wall. For example, the outer edge (e.g., outer diameter) of a flange may undulate (e.g., similarly to a sinusoidal wave) to maintain contact to a dynamic luminal wall, e.g., to a bowl-shaped antral wall.

When in position in a patient, a flange extends, wholly or partially, in a direction having a radial component from a longitudinal axis of the pyloric orifice (e.g., between 90° and 180° outward from the longitudinal axis of the pyloric orifice). Alternatively, the flange may extend at angle that varies along its radius. The angle may also vary in opposing directions over its radius. For example, a flange may become more acute at a central region and gradually become more obtuse (e.g., bowl-shaped) at a greater radius. It will be understood that, once attached to a proximally oriented surface proximal to the pyloric orifice, the angle of extension of the flange will substantially match that of the surface to that it is attached.

Suitable materials for flanges of the present invention include non-degradable polymers, such as polytetrafluoroethylene (PTFE), expanded PTFE (ePTFE), fluorinated ethylene propylene (FEP), perfluoroalkoxy (PFA), ethylene tetrafluoroethylene (ETFE), and polyvinylidene fluoride (PVDF). The flange can be an extension of the sleeve material (e.g., integrally formed), and it may have a greater thickness than all or a portion of the remainder of the sleeve. The thickness of the flange will depend on means of attachment to a patient.

A flange can be attached to a sleeve by any suitable means known in the art. In some cases, the proximal end of the sleeve is cut longitudinally at one or more points along its circumference, creating strips of sleeve material that can be splayed out and attached to a flange. In some embodiments, these portions of the sleeve can be sandwiched between two flange pieces. The two flange pieces (e.g., donut-shaped portions of PTFE/FEP) can then be attached to one another, for example, by hand soldering, to entrap the sleeve portions therebetween such that the sleeve lumen passes through the holes in the flange. In other embodiments, the sleeve and flange may be connected together by a biodegradable and/or bioresorbable polymer. In this configuration, the connection of the sleeve to the flange can have a controlled degradation, e.g., to facilitate the removal of the sleeve. The flange remains in place, and a new sleeve can be connected to the flange if necessary. Other methods of attaching the flange to the sleeve are known in the art.

### Sleeves

Gastrointestinal sleeves suitable for adaptation for the presently disclosed gastrointestinal devices are known in the art and described, e.g., in U.S. Pat. Nos. 7,025,791, 7,608,114, 7,695,446, 7,678,068, 7,122,058, 7,476,256, 7,815,589, 7,837,643, 8,057,420, 7,815,591, 7,771,382, and 7,766, 973, each of that is incorporated herein by reference.

Sleeves of the present device may be connected to a flange for attachment to a luminal wall proximal to the pyloric orifice (e.g., at a proximally oriented surface of the pyloric sphincter or the antrum of the stomach). As such, a sleeve is configured to receive fluid from the stomach as it moves past the flange, through the pylorus.

In general, sleeves of the gastrointestinal device are thin-walled, collapsible, flexible, and floppy (i.e., they do not support the entirety of their weight, for example, if stood on end, they would buckle). Thus, sleeves can reduce or eliminate contact of fluid with walls of the intestine or digestive solutions secreted therefrom while transmitting natural peristaltic forces to propel the fluid through the intestines. After fluid from the stomach has passed through the sleeve, the sleeve may become thin and floppy, permitting the sleeve to conform (i.e., contour to the shape of) to the inner walls of the intestine. In some cases, the sleeve is substantially non-compliant and drapes away from the intestinal walls, thereby permitting pancreatic juices to flow unimpeded into the duodenum through the ampulla of vater. Passage of fluid through the sleeve is also enhanced if the sleeve is made from a material having a low coefficient of friction (e.g., a static or kinetic coefficient of friction of less than about 0.3, e.g., about 0.29, about 0.28, about 0.27, about 0.26, about 0.25, about 0.24, about 0.23, about 0.22, about 0.21, about 0.2, about 0.19, about 0.18, about 0.17, about 0.16, about 0.15, about 0.14, about 0.13, about 0.12, about 0.11, about 0.1, or less).

Such material properties can be found in a sleeve formed from ePTFE, or from a combination with another material. For example, one such combination includes an ePTFE layer of material combined with a different fluoropolymer layer, such as FEP. The combination of the FEP with ePTFE provides a low coefficient of friction while also being substantially non-permeable. In some embodiments, another material such as PTFE is applied to an ePTFE substrate using vapor deposition. Alternatively or in addition, the sleeve can be formed using polyolefin films, such as low density polyethylene (LDPE), high density polyethylene (HDPE), and polypropylene. Other materials suitable for use as part of a sleeve include cast polytetrafluoroethylene (e.g., TEFLON™), cast PTFE with FEP or PFA coating on a PTFE to minimize pin holes, extruded FEP and extruded PFA. These materials are solid and substantially non-porous in contrast to ePTFE, which is generally porous. Alternatively or in addition, the sleeve may be made from a material that has microbial resistance, or the sleeve may have a surface coating of an antimicrobial agent.

In some cases, the wall thickness of the sleeve is less than about 0.0025 inches (e.g., between 0.0003 and 0.0025 inches, e.g., from 0.0003 to 0.0010 inches, from 0.0010 to 0.0015 inches, from 0.0015 to 0.0020 inches, or from 0.0020 to 0.0025 inches, e.g., about 0.001 inches).

The length of the sleeve ranges from about 1 foot to about 5 feet (e.g., about 30 cm to about 150 cm). In some cases, the length of the sleeve is from 1 to 3 feet (e.g., 30 cm to 90 cm) from its proximal end (e.g., at flange) to its distal end (e.g., below the ligament of Treitz). In some embodiments, the sleeve has a length of 12 inches, 13 inches, 14 inches, 15 inches, 16 inches, 17 inches, 18 inches, 19 inches, 20 inches, 21 inches, 22 inches, 23 inches, 24 inches, 25 inches, 26 inches, 27 inches, 28 inches, 29 inches, 30 inches, 31 inches, 32 inches, 33 inches, 34 inches, 35 inches, or 36 inches, e.g., about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, about 100 cm, about 110 cm, about 120 cm, about 130 cm, about 140 cm, or about 150 cm. The length of the sleeve can be selected to bypass the duodenum and a portion of the jejunum. The length may be increased to further decrease absorption by bypassing a longer section of the jejunum. Thus, the length of the sleeve is variable and may dependent on the patient's height, weight, or body mass index.

The sleeve can have a diameter similar to that of a normal patient's intestine, e.g., at the duodenum or jejunum. Additionally or alternatively, the diameter (e.g., maximum diameter) of the sleeve can be from 0.5 to 3 inches (e.g., from 1.0 to 2.0 inches, e.g., about 1.5 inches). By maximum diameter, it is meant the diameter when the sleeve is open and has a substantially circular cross-section.

In some cases, the invention provides eversion resistant sleeves. Eversion resistant sleeves refer to sleeves that resist proximal eversion (e.g., aberrant proximal movement through the anchor and/or pyloric orifice that may cause obstruction to the flow of fluid). Eversion resistant sleeves may be made from a thickening of the sleeve material, e.g., at a portion of the sleeve distal to the flange, for example, as described in U.S. Pat. No. 7,766,973, herein incorporated by reference.

### Methods of Delivering a Gastrointestinal Device

Featured herein are methods for delivering a gastrointestinal device within the gastrointestinal tract of a patient using any of the delivery systems described herein. In particular, the methods include providing a container assembly housing a plurality of actuator legs along its longitudinal axis, wherein the actuator legs further include an anchoring system for a gastrointestinal device having a flange connected to a gastrointestinal sleeve at or near its proximal end, directing the container assembly into the patient's gastrointestinal tract, opening the actuator legs outward about the longitudinal axis proximal to the pylorus, ejecting the flange from the container assembly, and securing the flange to a luminal wall of the gastrointestinal tract.

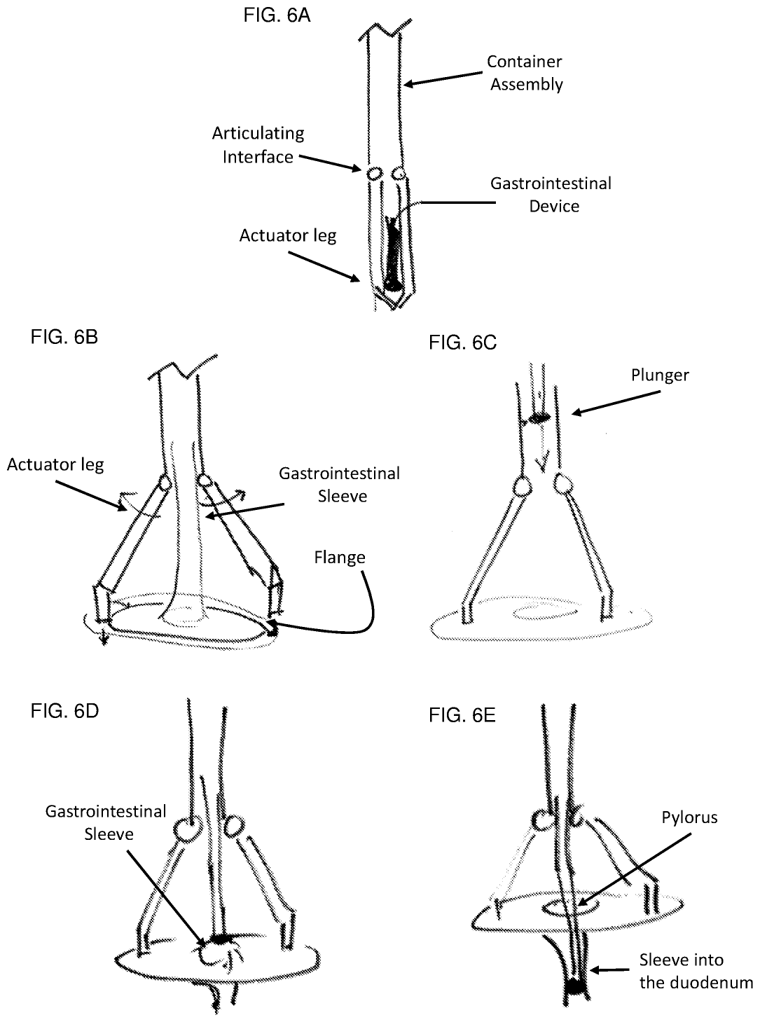

The invention further provides a method for delivering a gastrointestinal device within the gastrointestinal tract of a patient by providing a container assembly with the actuator legs connected to the distal end of the container assembly by an articulating interface. The actuator legs further include an anchoring system for a gastrointestinal device having a flange connected to a gastrointestinal sleeve at or near its proximal end, directing the container assembly into the patient's gastrointestinal tract, opening the actuator legs outward about the longitudinal axis proximal to the pylorus, ejecting the flange from the container assembly, and securing the flange to a luminal wall of the gastrointestinal tract. A pictorial example of the method is shown in FIGS. 6A-6E.

The container assembly may house all or a portion of a gastrointestinal device, e.g., the flanged gastrointestinal device and anchoring system described above. The device may packed, wholly or partially, within the container such that the pre-tethered flange and anchors are oriented at or near the distal end of the container with the remainder of the device, e.g., the sleeve, everted within the container assembly body. The tissue penetrating projection of each anchor may be oriented such that the one or more of the anchor's tissue retention elements are positioned proximal to the pylorus. The flange may be positioned across the pylorus and antrum and further secured by deploying the anchors from within or at the distal end of the container assembly.

Delivery of gastrointestinal devices such as gastric sleeves are typically installed using an endoscope and a guidewire. An endoscopic procedure consists of directing a length of guidewire through the working lumen of an endoscope and into the proximal portion of the duodenum. An example of a suitable guidewire consists of a 13-foot length of super-stiff 0.035″ monofilament stainless steel wire. A delivery catheter can be then directed through the pylorus and into the duodenum. The leading or distal end of the catheter can be attached, assembled to, or include a capsule or container defining a guidewire lumen along its side. The proximal end of the guidewire can be directed through the guidewire lumen, and the catheter can be advanced or directed along the guidewire to a point distal from the pylorus and into a desired position in the gastrointestinal tract (e.g., a position proximal to the pylorus). Other suitable guidewires are known in the art. Once a sufficient length of guidewire is in the optimal location, the endoscope may be removed, may remain in place, or be re-introduced at any point for delivery assistance.

Once the guidewire is in the desired location, the delivery system may be positioned proximal to the pylorus. The distal end of the catheter may be attached to, assembled to, or include a capsule assembly defining a guidewire lumen along its side or through its central longitudinal axis, as described herein. The guidewire may be directed through the guidewire lumen, and the capsule assembly can be advanced or directed along the guidewire. At this point, the clinician can utilize the camera of the endoscope to find the precise position desired proximal to the pylorus. Once the container assembly is at the desired location above the pylorus, the guidewire can be removed from the gastrointestinal tract. The delivery catheter may contain a plunger-based ejection mechanism through its central lumen that is slidable through the lumen along the longitudinal axis. Application of a distal force to the plunger moves it through the container assembly, pushing it to the distal edge and/or out of the container assembly.

The gastrointestinal device may be deployed using an ejection mechanism described herein. For example, a clinician can apply a distal longitudinal force to a proximal end of a pusher wire from the proximal end of the delivery catheter, to distally translate a plunger within the container assembly against the gastrointestinal device. The plunger exerts a force on the actuator legs that pushes them out of the container, allowing the actuator legs to outwardly open over a proximally oriented luminal surface positioning the flange of the gastrointestinal sleeve proximal to a proximally oriented luminal surface. The clinician can continue to apply force to the plunger to completely eject the remaining everted gastrointestinal device from the container assembly through the proximally oriented luminal surface and into the remainder of the gastrointestinal tract. In some cases, the force applied by the plunger to open the actuator legs is sufficient to allow for both the outward opening of the actuator legs (and thus the flange) over the proximally oriented luminal surface and insertion of the everted gastrointestinal sleeve into the gastrointestinal tract with no additional distal plunger force necessary. In other cases, the plunger outwardly opens the actuator legs by engaging the articulating interface the actuator legs are connected to at the distal end of the container assembly. The actuator legs open over a proximally oriented luminal surface, and the plunger can completely eject the remaining everted gastrointestinal device from the container assembly through the proximally oriented luminal surface and into the remainder of the gastrointestinal tract. In any of the above embodiments, natural peristaltic action can be used to further guide the remainder of the gastrointestinal device into the gastrointestinal tract beyond the pylorus (e.g., duodenum and beyond, e.g., the upper jejunum).

The flange can be attached to a proximally oriented luminal surface (e.g., a pyloric sphincter or the antral surface of the stomach) by transmission of a distal force from the anchors to the proximally oriented luminal surface. For example, the distal force can be directed (e.g., endoscopically) to the tissue penetrating projection of the anchor, to secure the flange to the luminal wall (e.g., by sequentially puncturing the luminal wall). The anchors may be contained within the lumen of the actuator legs, or alternatively, the anchors may be attached to the actuator legs using chemical or mechanical means. In some cases, the delivery system may have a second ejection system, e.g. a plunger, which contacts the anchors within the lumen of the actuator legs, and upon application of a distal force to the plunger, the anchors are released from the lumen of the actuator legs. Once the anchors are secured to a proximally oriented luminal surface (e.g., a pyloric sphincter or the antral surface of the stomach), the remainder of the device (e.g. the sleeve) can be delivered through the pylorus and into the duodenum, either through further application of the delivery catheter, e.g., a plunger, or through natural peristalsis.

### Methods of Treatment

Further provided herein are methods of treatment using a gastrointestinal device of the invention. In particular, the invention provides a method of treating a metabolic disorder by implanting a gastrointestinal device by attaching the flange to a proximally oriented luminal surface proximal to the pyloric orifice (e.g., a proximal surface of the pyloric sphincter or the antrum of the stomach) using a disintegrable anchor system. Metabolic disorders treatable by such methods include type 2 diabetes, NASH, NAFLD, obesity, and related comorbidities thereof. Any of the gastrointestinal devices described above, delivered and/or anchored by any suitable method described above, can be used to treat a metabolic disorder.

Gastrointestinal devices of the invention provide negative feedback within the enteric and/or nervous systems, reduced fat digestion, and reduced desire for food. Reduced fat digestion occurs because the sleeve delays the mixing of bile and pancreatic juices with chyme from the stomach until after the chyme leaves the sleeve. Reduced desire for food may occur because the sleeve reduces hormonal release from the duodenum. Additionally, providing poorly digested food to distal portions of the intestine, such as to the ileum, can trigger hormones that reduce appetite. Thus, such gastrointestinal devices can be used for treatment of various metabolic disorders (e.g., type 2 diabetes, NASH, NAFLD, and obesity) characterized by aberrant physiological response to ingested food, such as the incretin effect.

Placement of the gastrointestinal device may result in ingested food not digesting in a normal manner and modification of normal triggering of gut hormones. These hormones result in several physiology changes that impact hunger and digestion. Gut hormones that can be modified by devices of the invention include peptide YY (PYY), cholecystokinin (CCK) and ghrelin.

As under-digested food enters the ileum or distal part of the small intestine, PYY is released. PYY has been shown to have a direct effect on appetite, reducing it when released. Undigested food in the ileum indicates that too much food has been ingested. Thus, dependent on the length of the sleeve, the gastrointestinal device can promote deposition of undigested or partially digested food to the distal bowel. Therefore, the placement of a sleeve in the intestine promotes the delivery of undigested food to the ileum, which in turn promotes the release of PYY and reduces appetite in humans.

The hormone CCK is released when food contacts the duodenum. CCK triggers the release of bile from the gallbladder. Therefore, placing a sleeve in the duodenum reduces the release of CCK and thus reduces bile output resulting in reduction in the digestion of food.

Some ghrelin is released when food contacts the duodenum. Ghrelin has been shown to be a factor in the control of appetite. Gastrointestinal devices of the invention can reduce ghrelin output and thereby reduce appetite due to the bypass of the duodenum.

Type 2 diabetes is a disease of obesity that occurs when patients cannot adequately use the insulin they produce. Usually, it is not that the patient cannot make enough insulin, but rather that the patient's body cannot effectively use the insulin produced. A particularly dangerous result of type 2 diabetes is that blood sugar spikes after a meal. This is called post-prandial hyperglycemia. This spike in blood glucose causes cardiovascular and microvascular damage. One class of drugs used to control post-prandial hyperglycemia is the alpha-glucosidase inhibitors. These work by reducing the breakdown and absorption of carbohydrates to sugars. The gastrointestinal device has a similar function because it reduces bile and delays the breakdown and absorption of the carbohydrates, which are normally readily absorbed in the duodenum, but are less likely to be absorbed in the jejunum and ileum. Therefore, type 2 diabetes can be controlled by placing a sleeve in the proximal intestine to delay the digestion of carbohydrates which reduces post-prandial hyperglycemia.

A gastrointestinal device can be used to reduce type 2 diabetes symptoms by bypassing all or a portion of the duodenum. Following gastric bypass surgery, patients commonly experience complete reversal of type 2 diabetes. While the mechanism of this remarkable effect is not fully understood, the clinical result is reported in a high percentage of cases. Since the gastrointestinal devices described herein provide equivalent blockage of duodenal processes, a similar effect is elicited but without the trauma of surgery.

In the method of using the gastrointestinal device for treating diabetes, placement of the anchor within the stomach and/or duodenum allows the pylorus to operate normally. The length of the sleeve may be reduced to mimic the duodenum bypass. The sleeve may extends to just below the ligament of Treitz but may not extend further into the jejunum, thus allowing absorption to occur in the jejunum.

### Example

FIGS. 6A-6E provide an example of the delivery of a gastrointestinal sleeve and an anchoring system using an embodiment of a delivery system of the invention. To deliver the gastrointestinal device, e.g., the sleeve and flange, a delivery catheter with a container assembly connected to its distal end is positioned over the target area, e.g., proximal to the pylorus. FIG. 6A shows an embodiment of the container assembly with the gastrointestinal device everted within the container assembly. In this specific embodiment, the actuator legs of the container assembly are connected to the distal end of the container assembly with an articulating interface. However, it will be apparent to the skilled artisan that the actuator legs may be in any suitable configuration for delivering the device, e.g., within the container assembly. The actuator legs are closed while the container assembly is being positioned near the target area.

As described herein, the flange of the gastrointestinal device is pre-connected to the anchoring system using a series of anchors (e.g., disintegrable anchors) pre-strung with a disintegrable a tether. The anchors (e.g., disintegrable anchors), and therefore the flange of the gastrointestinal device, are releasably connected to the actuator legs of the delivery system. The specific embodiment from FIG. 6A shows two actuator legs in the delivery system, but the number of actuator legs may be as great as twelve actuator legs (e.g., three, four, five, six, seven, eight, nine, ten, eleven, or twelve).

When the container assembly is positioned at the desired target area, the actuator legs are opened by the operator. The opening of the actuator legs allows the flange, which is connected to anchors further connected to the actuator legs, to open over the target area, as is seen by the curved arrows through the actuator legs in FIG. 6B. The container assembly, with the flange opened over the target area, is further translated along the longitudinal axis of the delivery catheter to allow the disintegrable anchors connected to the actuator legs to insert into the pyloric sphincter, securing the flange to the pyloric sphincter.

The remainder of the everted gastrointestinal device, e.g., the sleeve, can be released from the container assembly using an ejection system. FIG. 6C shows a plunger having a round pusher plate contained within the lumen of the container assembly and an arrow indicating the direction of translation out of container assembly. As the plunger is translated out of the container assembly, shown in FIG. 6D, it passes through the distal end and out of the container assembly and to the pylorus. Once at the pylorus, the sleeve can be automatically or manually guided through the pylorus and into the remainder of the gastrointestinal tract, e.g., the duodenum and/or upper jejunum, by continued translation of the plunger shown in FIG. 6E. After the sleeve is inserted as far as the treatment procedure calls for, the plunger is retracted and the container assembly removed from the patient. Alternatively, the sleeve may be guided distally by normal peristalsis, such as is described in International Patent Application No. PCT/US2017/015658, which is incorporated herein by reference in its entirety. Other embodiments are within the claims.

### Other Embodiments

All publications, patents, and patent applications mentioned in this specification are herein incorporated by reference to the same extent as if each independent publication or patent application was specifically and individually indicated to be incorporated by reference. In the event of a conflicting definition between this and any reference incorporated herein, the definition provided herein applies.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure that come within known or customary practice within the art to which the disclosure pertains and may be applied to the essential features hereinbefore set forth, and follows in the scope of the claims.

What is claimed is:

1. A delivery system for placing a gastrointestinal device within the gastrointestinal tract of a patient, the delivery system comprising:
(a) a delivery catheter comprising a lumen defining a longitudinal axis, wherein the delivery catheter has a distal end and a proximal end;
(b) a container assembly connected to the distal end of the delivery catheter, the container assembly housing a plurality of actuator legs circumferentially arranged about the longitudinal axis, wherein each of the actuator legs includes an anchor; whereupon application of a force from the delivery catheter, the plurality of actuator legs opens outward; and
(c) a gastrointestinal device comprising: (i) a gastrointestinal sleeve comprising a proximal end and a distal end, the gastrointestinal sleeve configured to carry fluid from the proximal end to the distal end; and (ii) a flange connected to the gastrointestinal sleeve at or near the proximal end, the flange comprising a plurality of apertures through which the anchors are configured to pass to secure the proximal end of the sleeve within the gastrointestinal tract.

2. The delivery system of claim 1, wherein the gastrointestinal sleeve is everted such that the flange is at or near the distal end of the container assembly, and the remainder of the sleeve is loaded within the circumferential arrangement of actuator legs.

3. The delivery system of claim 1 or 2, wherein the disintegrable anchors are pre-assembled through the flange.

4. The delivery system of any one of claims 1-3, wherein the actuator legs are housed within the container assembly.

5. The delivery system of any one of claims 1-4, wherein the container assembly further comprises an ejection system whereupon distal displacement along the longitudinal axis, the actuator legs open outward about the longitudinal axis.

6. The delivery system of claim 5, wherein the ejection system is a plunger.

7. The delivery system of claim 6, wherein the plunger is configured to apply a distal longitudinal force on the actuator legs.

8. The delivery system of claim 6 or 7, wherein after outwardly opening the actuator legs, the plunger ejects the flange of the gastrointestinal device.

9. The delivery system of any one of claims 1-7, wherein the force generated by outwardly opening the actuator legs ejects the flange of the gastrointestinal device.

10. The delivery system of any one of claims 1-3, wherein the actuator legs are attached to distal portion of the container assembly by an articulating interface.

11. The delivery system of claim 10, wherein the articulating interface is selected from the group consisting of hinge, bearing, ball joint, spring, foam, elastomer, and sponge.

12. The delivery system of claim 10 or 11, wherein after outwardly opening the actuator legs, the plunger ejects the flange of the gastrointestinal device.

13. The delivery system of any of claims 1-12, wherein the actuator legs comprise a shape memory material.

14. The delivery system of claim 13, wherein the actuator legs comprise nitinol.

15. The delivery system of claim 14, wherein the actuator legs further comprises a physical stop such that the latitudinal dimension of the opening of the actuator legs does not exceed any latitudinal dimension of the flange.

16. The delivery system of any one of claims 1-15, wherein the actuator legs further comprise a lumen.

17. The delivery system of claim 16, wherein the disintegrable anchors are releasably housed within the lumen of the actuator legs.

18. The delivery system of any of claims 1-17, wherein the actuator legs further comprise an anchor ejection system configured to release the anchors.

19. The delivery system of claim 18, wherein the anchor ejection system is a plunger.

20. The delivery system of any of claims 1-15, wherein the anchors are connected to the actuator legs chemically.

21. The delivery system of any one of claims 1-15, wherein the anchors are connected the actuator legs mechanically.

22. The delivery system of any one of claims 1-21, wherein the force applied to each of the anchors from the delivery system is between about 0.1 and 10 Newtons (N).

23. The delivery system of any one of claims 1-22, wherein the flange is configured to be positioned proximal to the patient's pyloric orifice.

24. The delivery system of any one of claims 1-23, wherein the anchors are disintegrable.

25. The delivery system of claim 24, wherein the disintegrable anchors comprise a degradable material.

26. The delivery system of claim 25, wherein the disintegrable anchors comprises a degradable material selected from the group consisting of poly(lactic acid) (PLA), poly(glycolic acid) (PGA), poly(L-lactic acid) (PLLA), poly-LD-lactide (PLDLA), poly-DL-lactide (PDLLA), diolamine, trimethylene carbonate, caprolactone, dioxanone, polydioxanone (PDO), and copolymers thereof.

27. The delivery system of claim 26, wherein the disintegrable anchors comprises PLLA.

28. The delivery system of any one of claims 1-23, wherein the anchors are not disintegrable.

29. The delivery system of claim 28, wherein the anchors comprise a material selected from the group consisting of polyethylene terephthalate (PET), polyether ether ketone (PEEK), polyoxymethylene, polytetrafluoroethylene (PTFE), or a ceramic.

30. The delivery system of any one of claims 1-29, wherein the anchors are pre-strung with a disintegrable tether.

31. The delivery system of claim 30, wherein the disintegrable tether comprises the same material as the disintegrable anchors.

32. The delivery system of claim 31, wherein the disintegrable tether comprises PLLA.

33. The delivery system of claim 30, wherein the disintegrable tether comprises a different material than the anchors.

34. The delivery system of claim 33, wherein the disintegrable tether comprises a non-degradable material selected from the group consisting of stainless steel, CoCr alloys, nitinol, and tantalum.

35. The delivery system of claim 34, wherein the disintegrable tether comprises nitinol.

36. The delivery system of any one of claims 1-35, wherein the flange is circular.

37. The delivery system of any one of claims 1-35, the flange is non-circular.

38. The delivery system of any one of claims 1-37, wherein the outer diameter of the flange is at least 10% greater than the diameter of the sleeve.

39. The delivery system of any one of claims 1-38, wherein the diameter of the sleeve is substantially constant along its length.

40. The delivery system any one of claims 1-39, wherein the sleeve is at least 30 cm in length.

41. A method of delivering a gastrointestinal device to the gastrointestinal tract of a patient, the method comprising the sequential steps of:
(a) providing a delivery system comprising a container assembly connected to a delivery catheter, the delivery catheter defining a longitudinal axis, the container assembly housing a plurality of actuator legs about the longitudinal axis, wherein the actuator legs further comprise an anchoring system for anchoring a gastrointestinal device comprising a flange at its distal end;
(b) directing the container assembly into the patient's gastrointestinal tract to a point proximal to the pylorus using the delivery catheter;
(c) opening the actuator legs outward about the longitudinal axis to position the anchoring system proximal to the pyloric sphincter; and
(d) ejecting the anchoring system from the actuator legs to secure the gastrointestinal device to the luminal wall.

42. The method of claim 38, wherein step (c) comprises using an ejection system.

43. The method of claim 39, wherein step (d) comprises using the ejection system.

44. The method of any one of claims 38-40, wherein the ejection system comprises a plunger.

45. The method of claims 38-41, wherein step (d) comprises passing the plunger through the actuator legs.

46. The method of any one of claims 38-42, wherein step (d) comprises ejecting the flange of the gastrointestinal device using the plunger.

47. The method of any one of claims 38-41, wherein step (d) comprises ejecting the flange from the container assembly using the force generated from outwardly opening the actuator legs.

48. The method of any one of claims 38-44, wherein the actuator legs comprise a shape memory material.

49. The method of claim 45, wherein the actuator legs comprise nitinol.

50. The method of any one of claims 38-46, wherein the anchoring system comprises anchors.

51. The method of claim 50, wherein the anchors are disintegrable.

52. The method of any claim 51, wherein the disintegrable anchors comprise a degradable material.

53. The method of claim 52, wherein the disintegrable anchors comprises a degradable material selected from the group consisting of PLA, PGA, PLLA, PLDLA, PDLLA, diolamine, trimethylene carbonate, caprolactone, dioxanone, PDO, and copolymers thereof.

54. The method of claim 53, wherein the disintegrable anchors comprises PLLA.

55. The delivery system of claim 50, wherein the anchors are not disintegrable.

56. The delivery system of claim 55, wherein the anchors comprise a material selected from the group consisting of polyethylene terephthalate (PET), polyether ether ketone (PEEK), polyoxymethylene, polytetrafluoroethylene (PTFE), or a ceramic.

57. The method of any one of claims 50-56, wherein the anchors are pre-strung with a disintegrable tether.

58. The method of claim 57, wherein the disintegrable tether comprises the same material as the disintegrable anchors.

59. The method of claim 58, wherein the disintegrable tether comprises PLLA.

60. The method of claim 57, wherein the disintegrable tether comprises a different material than the anchors.

61. The method of claim 60, wherein the disintegrable tether comprises a non-degradable material selected from the group consisting of stainless steel, CoCr alloys, nitinol, and tantalum.

62. The method of claim 60 or 61, wherein the disintegrable tether comprises nitinol.

63. The method of any one of claims 41-62, wherein the flange is circular.

64. The method of any one of claims 41-62, wherein the flange is non-circular.

65. The method of any one of claims 41-64, wherein the outer diameter of the flange is at least 10% greater than the diameter of the sleeve.

66. The method of any one of claims 41-65, wherein the sleeve comprises a polymeric liner.

67. The method of claim 66, wherein the polymeric liner is selected from the group consisting of polytetrafluoroethylene (PTFE), expanded polytetrafluoroethylene (ePTFE), fluorinated ethylene propylene (FEP), perfluoroalkoxy (PFA), ethylene tetrafluoroethylene (EFTE), and polyvinylidene fluoride (PVDF).

68. The method of claim 67, wherein the polymeric liner is PTFE.

69. The method of any one of claims 41-68, wherein the diameter of the sleeve is substantially constant along its length.

70. The method of claims 41-69, wherein the sleeve is at least 30 cm in length.

71. A method of delivering a gastrointestinal device to the gastrointestinal tract of a patient, the method comprising the sequential steps of:
(a) providing a delivery system comprising a container assembly connected to a delivery catheter, the delivery catheter defining a longitudinal axis, the container assembly housing an anchoring system for anchoring a gastrointestinal device comprising a flange at its distal end;
(b) directing the container assembly into the patient's gastrointestinal tract to a point proximal to the pylorus using the delivery catheter; and
(c) ejecting the anchoring system from the container assembly to secure the gastrointestinal device to the luminal wall.

72. The method of claim 71, wherein the container assembly further comprises a plurality of actuator legs along its longitudinal axis.

73. The method of claim 72, wherein the actuator legs are attached to the distal portion of the container assembly by an articulating interface.

74. The method of claim 73, wherein the articulating interface is selected from the group consisting of hinge, bearing, ball joint, spring, foam, elastomer, and sponge.

75. The method of any one of claims 71-74, wherein step (c) is performed using an ejection system.

76. The method of claim 75, wherein the ejection system is a plunger.

77. The method of any one of claims 71-76, wherein step (c) comprises distally displacing the plunger to open the actuator legs outwardly about the longitudinal axis of the container assembly.

78. The method of any one of claims 71-77, wherein step (b) is performed using a delivery catheter.

79. The method of any one of claims 71-78, wherein step (c) comprises ejecting the flange of the gastrointestinal device using the plunger.

80. The method of any one of claims 71-79, wherein the flange of the gastrointestinal device is guided to a luminal wall using the plunger.

81. The method of any one of claims 71-80, wherein the luminal wall is the patient's pyloric sphincter.

82. The method of claim 72 or 73, wherein the anchoring system is releasably connected to the actuator legs.

83. The method of any one of claims 71-82, wherein the anchoring system comprises anchors.

84. The method of claim 83, wherein the anchors are disintegrable.

85. The method of claim 84, wherein the disintegrable anchors comprises a degradable material.

86. The method of claim 85, wherein the disintegrable anchors comprises a degradable material selected from the group consisting of PLA, PGA, PLLA, PLDLA, PDLLA, diolamine, trimethylene carbonate, caprolactone, dioxanone, PDO, and copolymers thereof.

87. The method of claim 86, wherein the disintegrable anchors comprises PLLA.

88. The delivery system of claim 83, wherein the anchors are not disintegrable.

89. The delivery system of claim 88, wherein the anchors comprise a material selected from the group consisting of polyethylene terephthalate (PET), polyether ether ketone (PEEK), polyoxymethylene, polytetrafluoroethylene (PTFE), or a ceramic.

90. The method of any one of claims 83-89, wherein the anchors are pre-strung with a disintegrable tether.

91. The method of claim 90, wherein the disintegrable tether comprises the same material as the disintegrable anchors.

92. The method of claim 91, wherein the disintegrable tether comprises PLLA.

93. The method of any of claim 90, wherein the disintegrable tether comprises a different material than the anchors.

94. The method of claim 93, wherein the disintegrable tether comprises a non-degradable material selected from the group consisting of stainless steel, CoCr alloys, nitinol, and tantalum.

95. The method of claim 94, wherein the disintegrable tether comprises nitinol.

96. The method of any one of claims 71-95, wherein the flange is circular.

97. The method of any one of claims 71-95, wherein the flange is non-circular.

98. The method of any one of claims 71-97, wherein the outer diameter of the flange is at least 10% greater than the diameter of the sleeve.

99. The method of any one of claims 71-98, wherein the sleeve comprises a polymeric liner.

100. The method of claim 99, wherein the polymeric liner is selected from the group consisting of PTFE, ePTFE, FEP, PFA, EFTE, and PVDF.

101. The method of claim 100, wherein the polymeric liner is PTFE.

102. The method of any one of claims 71-101, wherein the diameter of the sleeve is substantially constant along its length.

103. The method of any one of claims 71-102, wherein the sleeve is at least 30 cm in length.

104. A method of treating a metabolic disorder, the method comprising delivering a gastrointestinal device of any one of claims 1-40.

105. A method of treating a metabolic disorder, the method comprising delivering a gastrointestinal device using the method of any one of claims 41-103.

106. The method of claim 104 or 105, wherein the metabolic disorder is type 2 diabetes.

107. The method of claim 104 or 105, wherein the metabolic disorder is non-alcoholic steatohepatitis (NASH).

108. The method of claim 104 or 105, wherein the metabolic disorder is non-alcoholic fatty liver disease (NAFLD).

109. The method of claim 104 or 105, wherein the metabolic disorder is obesity.
